(19) United States
(12) Patent Application Publication
(10) Pub. No.: US 2010/0268110 A1
(43) Pub. Date: Oct. 21, 2010

(54) SYSTEMS AND METHODS FOR CHARACTERISTIC PARAMETER ESTIMATION OF GASTRIC IMPEDANCE SPECTRA IN HUMANS

(76) Inventors: Nohra E. Beltrán, Mexico City D.F. (MX); Jozefus J.M. De Folter, Uxbridge (GB); Maria M. Godinez, Mexico City D.F. (MX); Emilio Sacristán Rock, Mexico City (MX)

Correspondence Address: WILSON, SONSINI, GOODRICH & ROSATI, 650 PAGE MILL ROAD, PALO ALTO, CA, 94304-1050, US

(21) Appl. No.: 12/724,407

(22) Filed: Mar. 15, 2010

Related U.S. Application Data
(60) Provisional application No. 61/160,409, filed on Mar. 16, 2009.

(51) Int. Cl. A61B 5/053

(52) U.S. Cl. 600/547

(57) ABSTRACT

Impedance spectroscopy has been proposed as a method of monitoring mucosal injury due to hypoperfusion and ischemia in the critically ill. The invention includes an algorithm developed to calculate the characteristic electrical values that best describe human gastric impedance measurements and simplify the information obtained with this method. A database of gastric spectra was obtained from healthy volunteers, cardiovascular surgery and critically ill patients. The gastric spectrum forms two semi circles in the complex domain, divided into low frequency (F<10 kHz) and high frequency (F>10 kHz). A fitting algorithm was developed based on the Cole model, and central characteristic parameters were calculated. The parameters were validated using the normalized mean squared error and 0.7% of the spectra were discarded. From the experimental data obtained in humans, the greatest changes observed as the gastric mucosa becomes ischemic occur at low frequencies, which are specific and sensitive to tissue damage, and vary with the degree of hypoperfusion.

### CROSS-REFERENCE

This application claims the benefit of U.S. Provisional Application No. 61/160,409 filed Mar. 16, 2009, which application is incorporated herein by reference in its entirety.

### BACKGROUND OF THE INVENTION

Certain medical conditions can be monitored by measuring the impedance of a mammalian patient's tissue. This can be done by placing electrodes in contact with the tissue through which a low current can be passed through the tissue. It is known to use this technique for diagnostic and therapeutic applications. Electrical impedance spectroscopy (EIS) has been used, for example, for cellular measurements, volume changes estimation, body composition analysis, tissue classification and tissue monitoring. Impedance measurements can be used to detect cellular edema, and other events related to the metabolism of the tissue cells.

Electrical impedance spectroscopy measures the electrical impedance spectra of superficial tissues by placing an electrically conductive probe in contact with the tissue sample. Biological tissues have an electrical impedance which is dependant on the frequency of the current passed through the tissue. The biological tissues contain a number of components, such as a nucleus and a cytoplasm which have both resistive and capacitive properties. It is known, for example, that in cancerous and pre-cancerous tissues there is a significant change in the size of the cell nuclei, in the shape of the cells and in the arrangement of cells which form the tissue. These changes affect the electrical impedance of the tissue sample, so electrical impedance tomography can be used to detect significant changes in cell structure and therefore assist in providing a diagnosis for patients.

The magnitude of the electrical impedance, and the dependence of the electrical impedance on frequency of a tissue sample, have been found to be indicative of the tissue composition. It has been found that different tissue structures are associated with different frequency bands within an electrical impedance spectrum.

At low frequencies (less than about 1 kHz) the electrical current may be unable to pass through cells due to the capacitance of the cellular membrane resulting in charge accumulation at large membrane interfaces. At intermediate frequencies, such as in the region of about 1 kHz to 1 MHz (also known as the beta dispersion region), cell structures are the main determinant of tissue electrical impedance and current begins to penetrate the cell membranes. At higher frequencies (greater than about 1 MHz) the current is able to pass through the cells and the nuclei and at even higher frequencies (>1 GHz) the molecular structure is the determining factor contributing towards the electrical impedance of the tissue sample.

Measuring electrical current patterns produced by a particular tissue sample over a range of frequencies, and applying inverse modeling procedures, can determine a set of electrical parameters. Intracellular resistance of a given tissue sample can be significantly affected by the relative sizes of the nucleus and the cell. Therefore the electrical impedance of a tissue sample can be used to distinguish between tissues having different nuclear volume to cytoplasm volume ratios. Tissue samples having a higher ratio of nuclear volume to cytoplasm volume may be indicative of, for example, pre-cancerous tissues. The application of electrical impedance measurements using a probe which bears four electrodes on an end face in cervical cytology is disclosed in Electronics Letters, 36(25) 2060-2062 and in The Lancet, 355: 892-95, which are hereby incorporated by reference in their entirety. EIS is an emerging diagnostic method based on the study of passive electrical properties of biological tissues that can be applied to characterize these tissues. See, for example, U.S. Patent Pub 2008/0232675 for Apparatus For Measuring Tissue Samples Electrical Impedance by Brian Hilton Brown et al, which are hereby incorporated by reference in their entirety. This technique provides good information about tissue structure and it has been used in the measurement of changes in ischemia or perfusion in different organs. See, e.g., A. H. Kyle, C. T. Chan, A. I. Minchinton, “Characterization of three dimensional tissue cultures using electrical impedance spectroscopy,” Biophys J, vol. 76, pp. 2640-2648, 1999; E. Gersing, “Impedance spectroscopy on living tissue for determination of the state of organs,” Bioelectrochem. Bioenerg. vol 45, pp. 145-149, 1998; S. Kun, R. A. Peura, “Selection of measurement frequencies for optimum extraction of tissue impedance model parameters,” Med. Biol. Eng. Comput., vol. 37, pp. 699-703, 1999, which are hereby incorporated by reference in their entirety. Complex impedance spectroscopy also provides phase information, so that resistive and reactive tissue components can be separated, producing more reliable measurements. Gersing also used this method to assess levels of tissue damage in different organs.

A minimally invasive method of assessing the condition of the mucosa has been developed that measures the impedance spectrum of the mucosa. See, U.S. Pat. No. 6,965,795, which is hereby incorporated by reference in its entirety. Along with the technique, an impedance spectroscopy probe and nasogastric tube ISP/NGT allows the direct acquisition of an electric impedance spectrum of the mucosa, which can be used to identify and continuously monitor the WSGR Docket No. 37022-703.201 level of tissue damage. See U.S. Pat. No. 6,882,879, which is hereby incorporated by reference in its entirety.

Hypoperfusion and ischemia can cause changes in the impedance spectra of the gastric wall in cardiovascular surgery patients, proposing that this technology may be a useful prognostic and diagnostic monitoring tool. See N. Beltran, G. Sanchez-Miranda, M. Godinez, U. Diaz, E. Sacristán, “Gastric impedance spectroscopy in elective cardiovascular surgery patients,” Physiol Meas, vol. 27(3), pp. 265-277, 2006, which is hereby incorporated by reference in its entirety.

Some bioimpedance spectrometers measure resistance and reactance over a range of frequencies and, by application of a mathematical model for an equivalent circuit (Cole model), estimate a number of parameters. Some use fitting of experimental data to the model and others use measured impedances. See, e.g., S. Kun, B. Ristic, R. A. Peura, R. M. Dunn, “Algorithm for tissue ischemia estimation base on electrical impedance spectroscopy,” IEEE Trans. on Biomed Eng., vol. 50 (12), pp. 1352-1359, 2003; L. C. Ward, T. Essex, B. H. Cornish, “Determination of Cole parameters in multiple frequency bioelectrical impedance analysis using only the measurement of impedances,” Physiol Meas, vol. 27(9), pp. 839-850, 2007, which are hereby incorporated by reference in their entirety.

What is needed is an algorithm that can be applied to the collected mammalian data which can be used to obtain characteristic parameters from, for example, a measured gastric impedance spectra to provide a simplified data set.

### SUMMARY OF THE INVENTION

The invention is directed to systems and methods of characteristic parameter estimation of gastric impedance spectrum. The systems and methods may use an algorithm to obtain characteristic parameters from a measured gastric impedance spectra. The algorithm transforms measured data so the resulting data is simplified and diagnosis is easier to achieve. Various aspects of the invention described herein may be applied to any of the particular applications set forth below or for any other types of systems or methods for measuring bioelectric parameters. The invention may be applied as a standalone system or method, or as part of a diagnostic or treatment system. It shall be understood that different aspects of the invention can be appreciated individually, collectively, or in combination with each other.

One aspect of the invention may be directed to a system configured to determine characteristic parameter estimations of gastric impedance spectra. The system may include one or more monitoring device configured to measure at least one bioelectric parameter of a subject. The system may also include a memory including at least one physiological data profile with one or more electrical characteristic relating to a physical condition. The memory may be provided in a computer, or on one or more database. The system may also include a processor in communication with the monitoring device, wherein said processor receives the bioelectric data, transforms the bioelectric data using a model, such as a Cole model, to one or more characteristic electrical value, and compares said characteristic electrical value with the physiological data profile to determine whether the subject has the physical condition. The processor may be provided on a computer or other device.

Another aspect of the invention may be a method for determining characteristic parameter estimation of gastric impedance spectra. The method may include receiving, at a processor, a first number of electrical measurements from a monitoring device configured to collect the electrical measurements from a tissue of a subject. The method may also include transforming, at the processor, the electrical measurements based on a model to a reduced number of characteristic parameter, and comparing the characteristic parameters with one or more profiles based on data representing gastric mucosa under different conditions. The method may further include collecting electrical measurements from the monitoring device through the following steps: starting electrical current tissue excitation at the tissue of the subject using one or more electrodes, allowing a period of time to pass for a voltage signal to stabilize, writing voltage data to a first in, first out (FIFO) memory buffer, stopping electrical current tissue excitation, stopping writing to the FIFO memory buffer; and/or reading voltage data from the FIFO memory buffer.

A tangible computer usable medium may be provided in accordance with another aspect of the invention. The computer readable medium may have a computer readable program code embedded therein, said computer readable program code adapted to be executed to implement a method for determining characteristic parameter estimation of gastric impedance spectra. The method may include the steps of receiving a plurality of electrical measurements from a monitoring device configured to collect the electrical measurements from a tissue of a subject, wherein said electrical measurements include a tissue signal and a reference signal, obtaining a phase value from for a tissue signal and reference signal pair, obtaining an amplitude value for the tissue signal and reference signal pair, and calculating a resistance value and a reactance value from the phase value and the amplitude value.

Other goals and advantages of the invention will be further appreciated and understood when considered in conjunction with the following description and accompanying drawings. While the following description may contain specific details describing particular embodiments of the invention, this should not be construed as limitations to the scope of the invention but rather as an exemplification of preferable embodiments. For each aspect of the invention, many variations are possible as suggested herein that are known to those of ordinary skill in the art. A variety of changes and modifications can be made within the scope of the invention without departing from the spirit thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a Cole-Cole plot of impedance resembling a semi-circle in complex domain;

FIG. 2 is a Cole-Cole plot of the fitted model with 2 dispersion regions obtained in gastric tissue;

FIG. 3 is a Cole-Cole plot of the fitted model obtained from averaged spectra of healthy volunteers and cardiovascular surgery patients;

FIG. 4 is an overview of a system having a server, a CPU, a monitor, storage media, input devices, etc. upon which a software program performing the algorithm disclosed herein would be performed;

FIG. 5 is a graph which illustrates amplitude and phase values converted to resistance;

FIG. 6 is a graph which illustrates amplitude and phase values converted to reactance; and

FIG. 7 is a graph which the central points contained in a Nyquist graph for each semicircle.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The invention is directed to systems and methods of characteristic parameter estimation of gastric impedance spectrum. The systems and methods may use an algorithm to obtain characteristic parameters from a measured gastric impedance spectra. A monitoring device may be used to assist with measuring gastric impedance spectra, or other electrical or physiological parameters from a subject. The algorithm may transform measured data so the resulting data is simplified and diagnosis is easier to achieve. The measured parameters may be transformed to a reduced number of characteristic values. The characteristic values may indicate a physical condition of the subject, and may assist with diagnosing the subject.

### I. Methodology

A. Impedance Spectrometer

A monitoring device may be used to collect one or more physiological measurement from a subject. A subject may preferably be a human, or may be an animal. A subject may be a patient, who may be undergoing treatment or being diagnosed, or may be involved in clinical or pre-clinical trials. In preferable embodiments, electrical measurements, such as impedance, resistance, voltage, or current measurements, are collected from the subject. The monitoring device may directly contact a tissue of the subject or be in electrical communication with a tissue of the subject. The tissue of the subject may be gastric tissue of the subject. For example, the monitoring device may directly physically or electrically contact a gastric wall or gastric mucosa. The monitoring device may include one or more electrodes. The monitoring device may provide an excitation current to a tissue and/or measure electrical properties of the tissue.

In some embodiments, one, two, or more electrodes may provide an electrical excitation to a tissue, and one, two or more electrodes may measure electrical properties of the tissues. The same electrode or different electrodes may be used for excitation and measurements. In some embodiments tissue measurements and reference measurements may be taken. A tissue signal (such as an electrical tissue response) may correspond to a measurement (e.g., differential voltage) taken by a plurality of electrodes. A reference signal (such as an electrical reference resistance) may correspond to a measurement (e.g., voltage drop) across a reference resister when an electrical excitation current is applied. A monitoring device may be a spectrometer, or may incorporate the use of a spectrometer.

A spectrometer may generate an excitation current. For example it may generate an excitation current of 1 mA pp at 25 different frequencies in a 100 Hz to 1 MHz bandwidth. Alternatively, the spectrometer may generate an excitation current at other values, such as a current falling within the range of about 0.1 mA to 10 mA. For example the excitation current may be about 0.1 mA, 0.2 mA, 0.3 mA, 0.4 mA, 0.5 mA, 0.6 mA, 0.7 mA, 0.8 mA, 0.9 mA, 1.1 mA, 1.2 mA, 1.3 mA, 1.5 mA, 2.0 mA, 3.0 mA, 4.0 mA, 5.0 mA, 7.0 mA, or 10.0 mA. The excitation current may be generated at any number of frequencies. For example, one, two, or more frequencies may be used. In some embodiments, about 5 frequencies, 10 frequencies, 15 frequencies, 20 frequencies, 30 frequencies, 35 frequencies, 40 frequencies, 50 frequencies, 60 frequencies, 70 frequencies, or 100 frequencies or more may be used. In some embodiments, the frequencies may fall within any range, including but not limited to 50 Hz to 50 MHz, 100 Hz to 25 MHz, 150 Hz to 10 MHz, 200 Hz to 1 MHz, 250 Hz to 750 Hz, or 300 Hz to 500 Hz. The frequencies may or may not be evenly spaced apart.

Suitable spectrometers may include, for example, an experimental Nicolet 6700 spectrometer available in multiple spectral ranges (from far IR to UV-Vis). Four Ag electrodes located on the distal tip of an impedance spectroscopy probe and nasogastric tube (ISP/NGT) function as ionic to electronic current transducers, e.g., as described in U.S. Pat. No. 6,882,879, which is incorporated by reference in its entirety herein. Impedance spectra are obtained by making discrete frequency measurements of the amplitude and phase of an electrical tissue response, relative to an electrical reference resistance. For example, if 25 different excitation frequencies are investigated, then measurements, such as tissue and reference measurements, may be taken at each of the 25 frequencies. From these measurements the resistance and reactance can be calculated at each frequency, to be discussed in greater detail elsewhere.

B. Model & Parameter Calculation

In order to calculate the characteristic electrical values that best describe the gastric impedance measurements (instead of relying on any single measurement that may be noisy), a theoretical model based on the Cole equations was fitted. See, K. S. Cole, “Permeability and impermeability of cell membranes for ions,” Proc. Cold Spring Harbor Symp. Quant. Biol., vol. 8, pp. 110-122, 1940, which is hereby incorporated by reference in its entirety. A weighted least squared algorithm was used to obtain the coordinates of a circle. Complex impedance (Z) is described by:

Z=R_{∞}+jX (1)

Cole proposed an expression to describe a semi circle, taking frequency into account:

Z=R_{∞}+(R₀−R_{∞})(1+(jwt)^{α}) (2)

where R is the tissue resistance (real), X is the tissue reactance (imaginary), R₀ is the resistance at zero frequency, R_{∞} is the resistance at infinite frequency, τ is the characteristic time constant, and α is the measure of the semicircular arc's depression below the real axis (which is a value between 0 and 1). See J. J. Ackmann, M. A. Seitz, “Methods of complex impedance measurements in biological tissue,” Crit. Rev. Biomed. Eng., vol. 11, pp. 281-311, 1984, which is hereby incorporated by reference in its entirety.

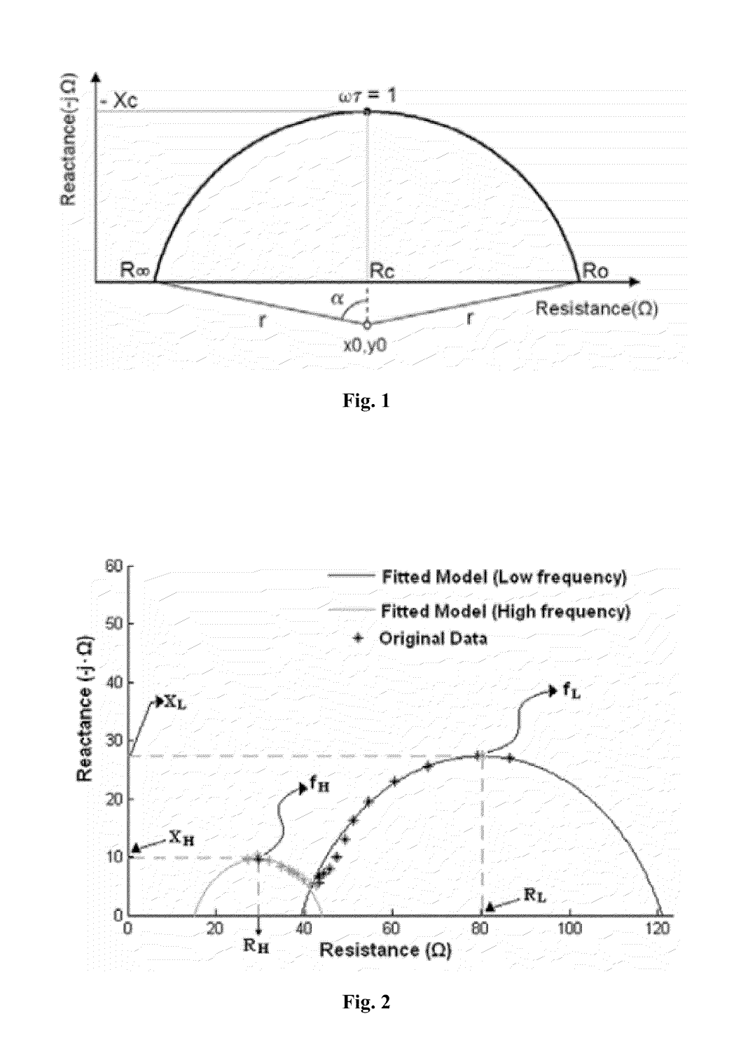

When tissular impedance is plotted in the complex domain, the resulting graph resembles a semi circle, which can be characterized as shown in FIG. 1, which is a Cole-Cole plot of impedance resembling a semi-circle in complex domain. The point at x₀, y₀ shows the center of the semi circle, with a radius of r. Central resistance, (R_{C}), reactance (X_{C}), and frequency (F_{C}) may be calculated where ωr=1.

The corresponding real and imaginary part of the top of each semicircle, give a characteristic value (also called ‘central point’) in resistance and reactance. Furthermore, when following the semi circle counter clockwise, the respective frequency of the plot increases. So the central point also has a respective frequency value.

FIG. 2 is a Cole-Cole plot of the fitted model with 2 dispersion regions obtained in gastric tissue. The original data may be provided, and one, two, or more semi-circles may be calculated or provided to fit the original data. The characteristic parameters may be obtained from the coordinates at the central frequency for each semi-circle. For example, for the first semi-circle on the left (for a high frequency fitted model), the central resistance R_{H}, reactance X_{H}, and frequency f_{H} may be calculated. Similarly, for the second semi-circle on the right (for a low frequency fitted model), the central resistance R_{L}, reactance X_{L}, and frequency f_{L} may be calculated.

Because gastric tissue impedance spectra may have two semi circles, Cole model parameters were calculated at two dispersion regions (low and high frequencies). Using a semi circle curve fitting algorithm, two semi circles can be found. Any curve-fitting algorithm, including algorithms discussed elsewhere herein, may be utilized. Any step provided by the algorithm may be directed by tangible computer readable media, code, instructions, or logic thereof. These may be stored in a memory, such as the memory of a computer or other device. The steps of the algorithm may be executed by a processor. In alternate embodiments, a semi circle curve-fitting algorithm may be used to find any number of semi circles based on the data collected. In some embodiments, the algorithm may automatically find one semi circle, two semi circles, three semi circles, four semi circles, or any predetermined number of semi circles based on the data collected. Alternatively, the algorithm may automatically fit the data to any number of semi circles, which need not be predetermined, but that may be determined using the algorithm to fit the data. The number of semi circles may correspond to classifications defining frequency ranges.

R_{∞}, R₀, and α are obtained from the centre and radius of each semi circle as:

R₀=x₀+\sqrt{r²−y₀²} (3)

R_{¢}=x₀+\sqrt{r²−y₀²} (4)

α=1−(2/π)arc sin(−y₀/r) (5)

The central points can be obtained as follows:

R_{C}=X₀ (6)

X_{c}=y₀−r (7)

Rewriting (2), taking the magnitude in order to convert to real values for τ gives:

τ=∥(R₀−Z)/(Z−R_{∞}))^{1/α}/(jw)∥ (8)

Using a model (e.g., provided by J. J. Ackmann, M. A. Seitz, “Methods of complex impedance measurements in biological tissue,” Crit. Rev. Biomed. Eng., vol. 11, pp. 281-311, 1984 which is incorporated by reference herein):

ωτ=1 (9)

or:

F_{c}=1/(2πτ) (10)

The central frequency (F_{c}) is estimated calculating the average r over all measured frequencies in the respective frequency range.

The gastric spectrum is divided into a low frequency (F<10 kHz approximately) and a high frequency range (F>10 kHz approximately). Using the described method, two central points are obtained. In other embodiments, the spectrum may be divided such that the dividing threshold between low and high frequencies may be at any other frequency, such as 1 kHz, 5 kHz, 15 kHz, 20 kHz, 50 kHz, or 100 kHz.

Any of these calculations may be conducted on a processor or using an algorithm. As previously discussed, any calculation or step provided by the algorithm may be directed by tangible computer readable media, code, instructions, or logic thereof, and may be executed by a processor (e.g., of a computer or other device).

C. Spectrum Reconstruction

Using (2) with the parameters obtained in the semi circle curve fitting (e.g., for low and for high frequency range), a ‘model’ spectrum can be reconstructed. These spectra are used to illustrate the model's behavior and to validate the measurements with the model using a normalized mean squared error. Alternative statistical methods for calculating deviations for validating the measurements may be used.

D. Impedance Measurements Characteristics

In some embodiments, data about various subjects may be collected. Such data may include electrical characteristics of the tissue, such as gastric tissue, of the subjects. In some embodiments, the collected data may be used to generate a physiological data profile. The data may be collected using a monitoring device. The data collected by the monitoring device may be analyzed using any of the techniques or algorithms described elsewhere herein.

In one embodiment, impedance spectra were measured in healthy gastric mucosa, in patients undergoing cardiovascular surgery, and in critically ill patients. Patients with massive gastrointestinal bleeding, esophageal obstruction, or nasopharyngeal obstruction; and volunteers with a history of gastrointestinal disease, previous abdominal surgery, gastrointestinal bleeding, and ongoing medical treatment, were not included in the studies. However, in some instances, data from such patients may be collected as well to assist with generating physiological data profiles.

An impedance spectrometry probe and nasogastric tube (ISP/NGT) was positioned in the stomach to measure tissue impedance. In alternative embodiments, the ISP/NGT may be positioned elsewhere in a subject and contact other tissue of the subject. ISP/NGT positioning was radiographically confirmed in all cases. In alternatively embodiments, the ISP/NGT placement may or may not be confirmed radiographically or by other techniques. Measurements obtained under improper ISP/NGT positioning or ISP/NGT conductivity failure were identified and excluded from the analysis.

A complete spectrum was obtained every minute. To reduce the effect of noise and motion artifacts, the spectra were averaged every ten minutes resulting in an averaged complete spectrum for every 10 minute window. In alternate embodiments, the complete spectrum may be obtained at any time interval (e.g., on the order of seconds, minutes, hours) and may be averaged at any time interval. In some embodiments, the measurements may be taken only once, a plurality of times, or periodically.

Cardiovascular surgery patients' data were used to characterize impedance spectral changes under differing degrees of hypoperfusion. The spectral changes were characterized as ischemia was progressing, and were used to evaluate the prognostic value of the parameters to post surgical ICU morbidity and mortality. Data may be collected from any subject with various physical conditions at different points in time to form a physiological data profile. Any collected data may be used to perform a reference point for electrical or other characteristics of tissues for particular physical conditions, such as ischemia or differing degrees of hypoperfusion.

Postoperative complication was defined as the presence of any of the following: Mechanical ventilation for more than 24 h, need of inotropic drugs for more than 48 h, or death. Measurements may or may not be taken under these conditions.

The intensive care patients' study was designed to obtain a database of human gastric impedance spectra under varied clinical conditions and pathologies. The parameter changes in these patients were used to evaluate their predictive value.

E. Statistical Analysis

The parameters were calculated for each average spectrum. The mean and the standard error (s.e) were calculated for each parameter and database. Sensitivity and specificity were calculated for each parameter. A Receiver Operating Characteristic (ROC) curve was constructed for each parameter to predict morbimortality of cardiovascular surgery patients, and to predict mortality of intensive care patients. The area under the ROC curve (AUC) must be greater than 0.5 for the parameter to have predictive value. A one sided z-test of significance was also performed for each ROC curve. Data is presented as mean s.e.

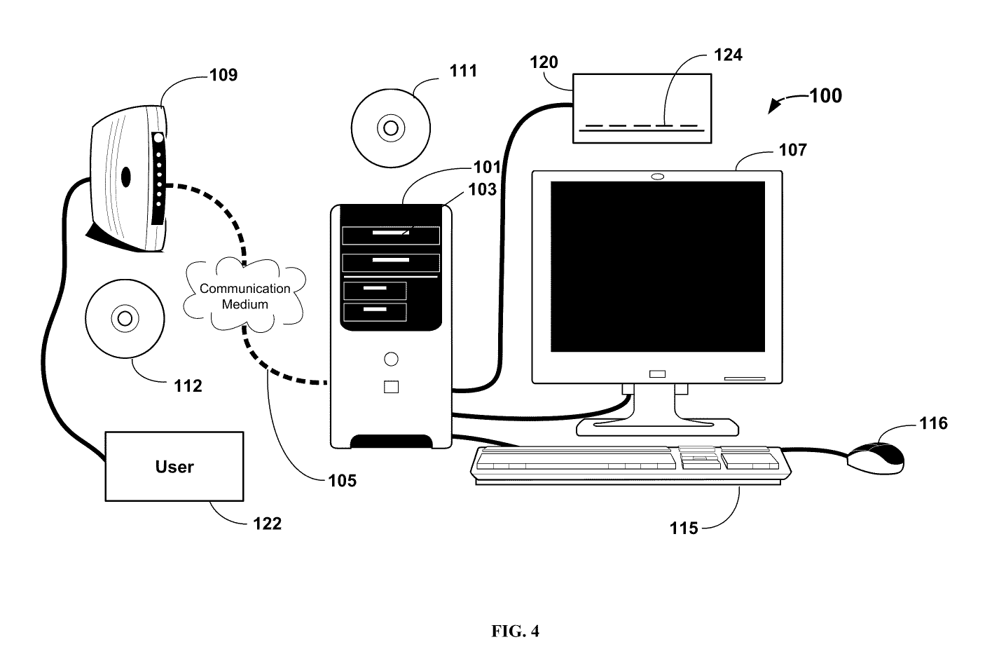

FIG. 4 is a diagram showing a representative example logic device through which reviewing or analyzing data relating to the present invention can be achieved. Such data can be in relation to a physiological parameter, or any other suitable parameter desired to be measured of a subject, such as a mammalian subject. A computer system (or digital device) 100 that may be understood as a logical apparatus that can read instructions from media 111 and/or network port 105, which can optionally be connected to server 109 having fixed media 112. The computer system 100 can also be connected to a network, such as the Internet, an intranet, or any other wide area or local area network. The system may include CPU 101, disk drives 103, optional input devices, illustrated as keyboard 115 and/or mouse 116 and optional monitor 107. Data communication can be achieved through the indicated communication medium to a server 109 at a local or a remote location. The communication medium can include any means of transmitting and/or receiving data. For example, the communication medium can be a network connection, a wireless connection or an internet connection. It is envisioned that data relating to the present invention can be transmitted over such networks or connections. The computer system can be adapted to communicate with a participant parameter monitor.

A user or participant 122 can also be connected to a variety of monitoring devices. The monitoring devices can be used to interact with the system. As will be appreciated by those skilled in the art, the computer system, or digital device, 100 can be any suitable device.

In one example, a subject may be in contact with a monitoring device. The monitoring device may include one or more electrodes that may be in electrical communication with a tissue of the subject, such as gastric tissue of the subject. The monitoring device may include a component that may generate an excitation signal and provide an excitation current to the tissue. The monitoring device may also include one or more probes that may measure an electrical property of the tissue. The probes may measure properties such as tissue measurements and reference measurements. The monitoring device may measure a physiological parameter that may be an electrical impedance measurement.

The monitoring device may communicate with a computer or other device, which may include a processor and a memory. In some embodiments, a memory within the system (which may or may not include a memory of the computer, other database, server, or monitoring device) may include at least one physiological data profile stored thereon. The physiological data profile may include data on one or more electrical characteristic relating to a physical condition of a subject. The data for the physiological data profile may have been collected from one or more subjects, or may be theoretical data that may be provided by a user of a system or generated by the system. In some instances, the physiological data profile may include tissue and reference measurements at one or more frequency. The physiological data profile may also include a reduced number of electrical characteristics derived from the measurements.

A processor may be in communication with the monitoring device and may perform one or more steps with measurements taken by the monitoring device. For example, if a monitoring device measures a bioelectric parameter, the processor may receive the bioelectric parameter, and transform the bioelectric parameter based on a model to one or more characteristic electrical value. In one example, the model may be a Cole model. The model may involve fitting semi circles or other curves to resistance-reactance data points at one or more frequency. The processor may also compare the characteristic electrical value with the physiological data profile to determine whether the subject has the physical condition. The processor may be provided on a computer, server, or other device.

In some embodiments, a display screen (e.g., a computer monitor, device screen, projector, or other user interface) may be provided, which may display one or more result of the data processing or comparison. In some embodiments, the system may also include a server that may be configured to receive the bioelectric parameter from the monitoring device, and configured to communicate with the processor over a network. In one example, the server may be provided as an intermediate device between the monitoring device and a processor. In some embodiments, measurements from a monitoring device may be communicated over a network to be processed.

### II. RESULTS

Impedance spectra were measured in healthy gastric mucosa of 17 volunteers (213 spectra), in 55 patients undergoing cardiovascular surgery (2512 spectra), and in 103 critically ill patients (13474 spectra). Thus, impedance spectra may be measured for a variety of subjects with different physical conditions.

Thirty two of cardiovascular surgery patients developed complications. Prolonged ischemia (>4 h) was observed in 23 patients, 19 of whom developed complications.

In the critically ill patients study, 76 patients survived and 27 died (26.2%). Nine were deaths during the impedance spectroscopy monitoring period.

FIG. 2 shows the Cole-Cole plot of the fitted model with 2 dispersion regions from the data obtained in the gastric wall. The characteristic parameters were calculated as presented in FIG. 1. Subsequently a spectrum reconstruction was made.

The parameters were validated using the normalized mean squared error. A high error (≧1) would indicate an inconsistency between the calculated parameters and the model. Only 0.7% of the spectra were discarded because of a high error.

With this process, the information from 46 measurements can be condensed to 6 characteristic parameters: R_{L} (central resistance at low frequency), R_{H} (central resistance at high frequency), X_{L} (central reactance at low frequency), X_{H} (central reactance at high frequency), f_{L} (central frequency at low frequency) and f_{H} (central frequency at high frequency).

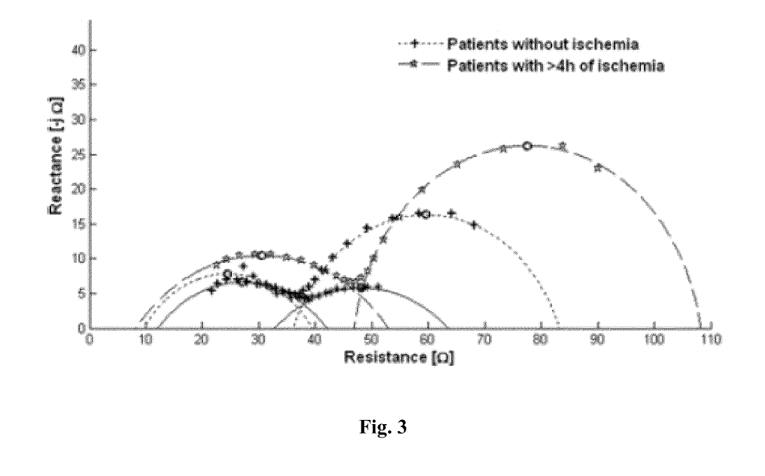

In order to show characteristic parameter differences between healthy and hypoperfused tissue, FIG. 3 shows the average spectra for 3 different groups: healthy volunteers (n=17), patients without ischemia or complications (n=5), and patients with prolonged ischemia and complications (n=19). Patients are from the cardiovascular surgery study. A Cole-Cole plot of the fitted model may have been obtained from the average spectra of the various subjects. Any number of semi circles may be provided. In one example, for each group, two semi circles may be provided (e.g., a low and high frequency semi circle for each group). Thus, when 3 different groups are investigated, six semi circles may be plotted based on the data collected.

TABLE I contains averaged values for central parameters calculated from three different studies. Low frequency parameters show higher variations in cardiovascular patients.

TABLE II shows area under the curve (AUC) (mean±s.e.) and p-levels calculated for characteristic parameters from the ROC analysis made to predict morbimortality of cardiovascular surgery patients. Low frequency parameters show better prediction of complications and death, in patients with a high degree of hypoperfusion.

TABLE III shows AUC (mean±s.e.) and p-levels calculated for the same parameters from the ROC analysis made to predict mortality of general intensive care patients.

TABLE I
CALCULATED MODEL PARAMETERS FOR
GASTRIC TISSUE MEASUREMENTS

| | | Cardiovascular | |
|---|---|---|---|
| Parameter | Healthy Volunteers | Surgery Patients | Critically ill Patients |
| R_{L} | 51.72 ± 0.71 | 69.57 ± 0.39 | 61.00 ± 0.13 |
| R_{H} | 28.47 ± 0.49 | 28.28 ± 0.13 | 30.84 ± 0.05 |
| X_{L} | 8.36 ± 0.22 | 20.86 ± 0.20 | 18.13 ± 0.08 |
| xₕ | 8.25 ± 0.17 | 9.18 ± 0.07 | 7.66 ± 0.03 |
| f_{L} | 1030 ± 102 | 524 ± 6.66 | 631 ± 5.68 |
| f_{H} | 5.9E+05 ± 47000 | 6.9E+5 ± 18000 | 3.15E+05 ± 786 |

TABLE II
AREA UNDER THE ROC CURVE FOR
CARDIOVASCULAR SURGERY PATIENTS
AS A PREDICTOR OF DEATH AND COMPLICATIONS

| Parameter | AUC | P level | n |
|---|---|---|---|
| X_{L} | 0.767 ± 0.061 | 0.001* | 55 |
| R_{L} | 0.742 ± 0.065 | 0.002* | 55 |
| X_{H} | 0.652 ± 0.072 | 0.033* | 55 |
| f_{L} | 0.601 ± 0.078 | 0.118 | 55 |
| R_{H} | 0.581 ± 0.075 | 0.155 | 55 |
| f_{H} | 0.528 ± 0.078 | 0.36 | 55 |

TABLE III
AUC FOR INTENSIVE CARE PATIENTS
AS A PREDICTOR OF DEATH

| Parameter | AUC | P level | 11 |
|---|---|---|---|
| R_{L} | 0.722 ± 0.105 | 0.049* | 103 |
| X_{L} | 0.683 ± 0.094 | 0.041* | 103 |
| R_{H} | 0.648 ± 0.088 | 0.087 | 103 |
| X_{H} | 0.634 ± 0.103 | 0.124 | 103 |
| f_{H} | 0.562 ± 0.073 | 0.206 | 103 |
| f_{L} | 0.544 ± 0.081 | 0.301 | 103 |

WSGR Docket No. 37022-703.201

### III. DISCUSSION

An algorithm was applied to the data to transform the data in order to calculate the characteristic electrical values that best describe the gastric impedance measurements, based on the Cole model. With the algorithm developed it is possible to condense the information from 46 measurements to 6 characteristic parameters. In some embodiments, any number of measurements may be taken. For example 5 or more, 10 or more, 15 or more 20 or more, 25 or more, 30 or more, 35 or more, 40 or more, 45 or more, 47 or more, 48 or more, 50 or more, 55 or more, 60 or more, 70 or more, 80 or more, or 100 or more measurements may be taken. In some instances, the measurements may be condensed to any number of characteristic parameters. For example, they may be condensed to 2 or fewer, 3 or fewer, 4 or fewer, 5 or fewer, 6 or fewer, 7 or fewer, 8 or fewer, 9 or fewer, 10 or fewer, 12 or fewer, 15 or fewer, 20 or fewer, 25 or fewer, 30 or fewer, or 50 or fewer characteristic parameters. The algorithm's estimation accuracy is high (only 0.7% error), and the characteristic parameters could be used for ICU monitoring, reducing the information obtained with the proposed technique. In some embodiments, the algorithm's estimation may yield 5% or less error, 3% or less error, 2% or less error, 1% or less error, 0.8% or less error, 0.7% or less error, 0.6% or less error, 0.5% or less error, 0.4% or less error, 0.2% or less error, 0.1% or less error, 0.05% or less error, or 0.01% or less error. The parameters calculated are influenced by ischemia and hypoperfusion as can be seen in FIG. 3.

In the cardiovascular group of patients, most complications can be associated with circulatory problems that should be reflected by poor gastric perfusion. In the general intensive care group of patients, the target population had a broad range of diagnoses, and some complications and deaths were not associated with gastric hypoperfusion.

From the experimental data obtained in humans by our research group, the greatest changes observed, as the gastric mucosa becomes ischemic, occur in the resistance and reactance at low frequencies. These two parameters correlate very well in fact, but the reactance is more consistent and is the electrical parameter expected to be most sensitive to tissue injury. Changes in reactance at high frequencies seem to occur more slowly and do not correlate well with changes at low frequencies, and probably reflect other tissue changes that may provide additional information, whereas resistance at high frequencies is almost constant under all circumstances and therefore provides no useful information. The central frequencies also change with ischemia (a shift toward lower frequencies can be seen) but are less sensitive than reactance.

The results presented indicate that low frequency resistance and reactance are the most descriptive parameters, which probably reflect tissue edema caused by prolonged ischemia, causing a net increase in intracellular to extracellular volume ratio.

Gastric impedance measurements are reproducible under clinical conditions, and good parameter estimation of those measurements was obtained through the developed algorithm. Analysis of the clinical results showed that the gastric tissue impedance model with 6 parameters, describes the behavior of the complete spectrum obtained from each patient and healthy volunteer. Low frequency resistance and reactance are sensitive to tissue damage, which varies with the degree of hypoperfusion, and show significant predictive values that may be valuable to clinicians.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### IV. Spectrum Measurements Done by the Hardware.

Steps of the spectrum measurements done by the hardware may include, but are not limited to the following

Reset first in, first out memory buffers (FIFOS)

Program frequency synthesizers

Start electrical current tissue excitation (sinusoidal)

Wait for voltage signal to stabilize

Start writing signal from analog-to-digital (A/D) converter to FIFOS

Stop electrical current tissue excitation

Stop writing to FIFOS

Read voltage data from FIFOS

This process may be repeated for each frequency (e.g., a spectrum of 25 frequencies in total). Just before this entire batch starts, the relays connected to the electrodes are activated, and deactivated again after finishing the entire spectrum. For each frequency, a time period of 16 voltage cycles is measured. This means that each voltage signal contains 16 sine waves. There are a total of 512 samples, so each digitized sine wave is represented by 32 samples. In other examples, other time periods may be used (e.g., 2 voltage cycles, 4 voltage cycles, 8 voltage cycles, 12 voltage cycles, 20 voltage cycles, 24 voltage cycles, etc.). Any number of samples may be used as well.

In some instances, the same process may be repeated for each frequency of the spectrum. In alternate embodiments, the processes may vary depending on the frequency. An entire spectrum of frequencies may or may not be repeated. When repeating a spectrum of frequencies, they may or may not utilize the same process or different values at the process.

Voltage data is stored in two channels: the tissue and the reference signals. The reference signal corresponds to the voltage drop across the reference resistor when the electrical current (excitation) is applied, and the tissue signal corresponds to the differential voltage of the tissue as measured at the two central probe electrodes.

The data can be written directly from the A/D converter to the FIFOS. After recording the signals, the data is transferred from the FIFOS to the internal memory and then be processed by the software.

The signal data may be validated. In one instance, the signal data is validated in the following two ways.
1. The tissue and reference signals are checked for value changes
2. The reference signal is checked for minimum amplitude (only the last half of the recorded signal)

The first check counts the number of times each signal changes value. Naturally each signal should describe a sine wave, hence having many changes over its 512-sample array. If the number of changes is 3 or less, it means there is an error in the FIFO input-output (I/O). The tissue and reference signals may be checked for the magnitude of value changes and/or the number of value changes. They may be compared to a threshold amount of change or number of changes. For example, a check may verify that that each signal changes 1 or less times, 2 or less times, 3 or less times, 4 or less times, 5 or less times, 6 or less times, 8 or less times, 10 or less times, or 15 or less times. The threshold amounts or numbers may depend on the sample size or may be fixed. In some instances, they may be predetermined, set by the user, or automatically generated. The signal value changes may or may not be tracked to determine whether the signals describe a sinusoidal wave.

The second check can provide an indication of whether an actual excitation frequency was sent to the probe. The amplitude of the reference signal may be constant regardless of the tissue measurement (unlike the amplitude of the tissue signal). If the peak-to-peak amplitude of this reference signal is very small, it means there was a problem with the excitation signal. In some instances, the amplitude of the reference signal may be compared to a threshold value to determine whether it is large enough. The threshold value may be predetermined, set by the user, or automatically generated. If at least one of the checks fails, the measurement is repeated (only the current frequency). If the measurement keeps failing, the entire spectrum is discarded.

After validation, the spectrum is processed.

A. The spectrum processing starts, using tissue and reference signals of, for example, the frequencies.

B. The measurements can be filtered to remove any noise from the signal.

The first processing step is provided by a digital filter that removes any noise from the signal. For this purpose, a unity-gain, 6th-order Chebyshev type II narrow bandpass filter may be used on each 16-cycle signals. Other types of filters known or later developed in the art may be used to remove noise from the signal (e.g., other digital filters, analog filters, linear filters, Butterworth filters, elliptic filters, Bessel filters, Comb filters, Chebyshev type I filter). Normalized stop band frequencies, where 1 corresponds to the Nyquist or “foldover” frequency (i.e., half the sampling rate), are 0.042 and 0.092. Therefore, at the normalized stop bands, i.e. from 0 to 0.042 and from 0.092 to 1, minimum filter attenuation is 40 dB. In other words, this filter allows passing of a narrow bandwidth centered on the excitation frequency, and subsequently rejects all other frequencies. Since sampling frequency may always be 32 times the excitation frequency, the same filter may be applied to every digitized tissue and reference signal regardless of the excitation frequency. In other instances, the sampling frequency may be a different value relative to the excitation frequency (e.g., N times the excitation frequency where N is any real number), or may vary relative to the excitation frequency.

The filter may be described by two vectors a and b (filter coefficients) and by the following differential equation:

y[0]=b[0]*x[0]+b[1]*x[1]+b[2]*x[2]+b[3]*x[3]+b[4]*x[4]+b[5]*x[5]+b[6]*x[6]−a[1]*y[1]−a[2]*y[2]−a[3]*y[3]−a[4]*y[4]−a[5]*y[5]−a[6]*y[6]

The filter may have any value for the filter coefficients. In other embodiments, other filters may be used which may have different characterizations.

C. Amplitude and Phase are Calculated for Each Frequency.

i. Phase

Measurements may be taken at one or more frequency, e.g., by the monitoring device. Such measurements may include a tissue signal and a reference signal for a frequency.

For each tissue and reference signal pair, the phase is obtained using, for example, a cross-correlation method over the last 8 cycles (the 2nd half of the entire signal). Cross-correlation searches for the optimum match position. For each sample, the measured signal data is multiplied with a ‘reference’ sine wave that has the same frequency as the signal (each sine wave is preferably always 32 samples long). All these products are finally added up into a sum. The reference sine wave is gradually moved in phase. So for each phase position, the sum of all the products of the signals is calculated. The optimum is easily found by the largest sum. Because of the nature of this method, it can be applied over a single sine wave cycle, or over various (8 in this case). This calculation may take a long time to process. Therefore, the method is applied in 3 runs, each refining in increased precision. This greatly increases performance. Thus, an iterative process may be used. The steps are as follows:

32 steps of 11.25 degrees (covering the entire 360 degrees) 20 steps of 1.125 degrees (covering 22.50 degrees)

30 to 40 steps of 0.1 degree (covering 3 to 4 degrees)

The number of steps is a bit more than strictly necessary to make sure the entire range of each previous precision is covered. So the first run gives a phase with a precision of 11.25 degrees, the second with a precision of 1.125 degrees and the last with a precision of 0.1 degrees. This gives a total of around 80 steps in stead of a normal 3600 steps (it would take in a single run with steps of 0.1 degrees) to get to 0.1 degrees precision.

In alternate implementations, an optimum match position may be calculated in any other manner. Or a cross-correlation search may be used with other parameters. Any number of sine wave cycles may be used (e.g., 1, 2, 3, 4, 5, 6, 7, 8, 9, 10, 11, 12, 13, 14, 15 or more). Any number of runs or iterations may be used (e.g., 1, 2, 3, 4, 5, 6, 7, 8, 9, 10 or more). Preferably, the number of steps may be 10 or fewer, 20 or fewer, 30 or fewer, 40 or fewer, 50 or fewer, 60 or fewer, 70 or fewer, 80 or fewer, 90 or fewer, 100 or fewer, 120 or fewer, 150 or fewer, 200 or fewer.

The actual phase difference is then calculated as: tissue phase minus reference phase.

ii Amplitude

The amplitude is calculated for each of the last 8 cycles (sine waves), by simply calculating the top-top difference. These 8 amplitude values are then averaged to a single value. Like the phase, the amplitude is calculated for both the tissue and the reference signal.

The actual amplitude is then calculated as: tissue amplitude divided by reference amplitude, multiplied by the reference resistance.

amp=(tisAmp/refAmp)*47
Where:
amp=actual amplitude
tisAmp=tissue amplitude
refAmp=reference amplitude

In alternate embodiments, the amplitude value may be collected and not averaged, or may be calculated for any number of the last cycles (e.g., last cycle, last 2 cycles, last 3 cycles, last 4 cycles, last 6 cycles, last 8 cycles, last 10 cycles, last 12 cycles, etc.). The amplitude values may be averaged to a single value, or any other statistical analysis may be performed to provide a representative value (e.g., median, mode).

D. Resistance and Reactance values.

The amplitude and phase values may be stored and converted to resistance and reactance values as well.

The polar coordinates of amplitude and phase are used, in which the real part is resistance and the imaginary part is reactance.

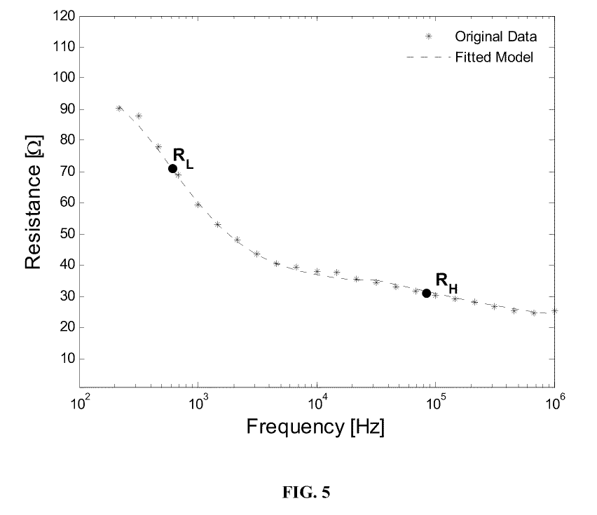

FIG. 5 is a graph which illustrates amplitude and phase values converted to resistance. The values derived from the original data are provided as well as a fitted model. Resistance may be plotted for a range of frequencies. For example, resistance may be calculated for each frequency data is collected within a spectrum (e.g., 100 Hz to 1 MHz). In some instances, resistance at a lower frequency may be higher than resistance at a higher frequency.

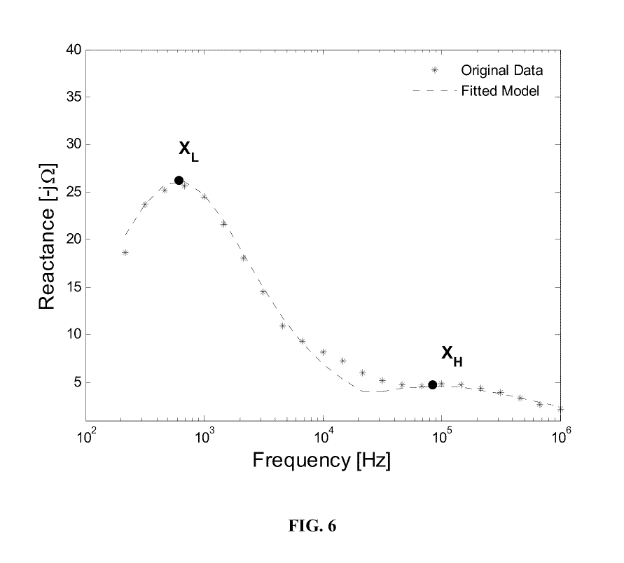

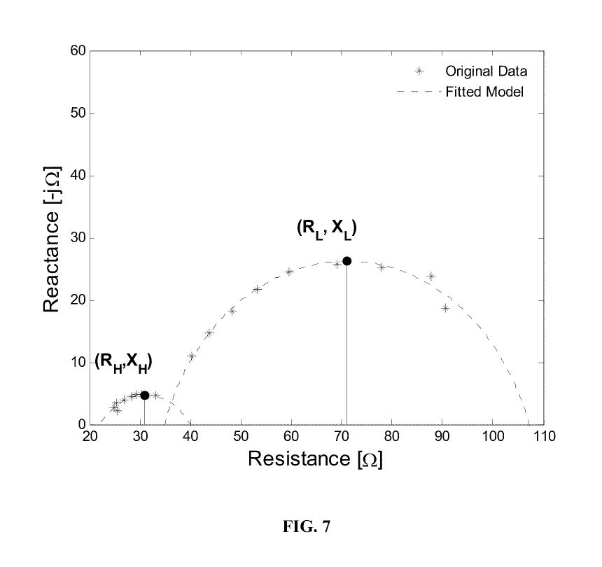

FIG. 6 is a graph which illustrates amplitude and phase values converted to reactance. The values derived from the original data are provided as well as a fitted model. Reactance may be plotted for a range of frequencies. For example, reactance may be calculated for each frequency data is collected within a spectrum (e.g., 100 Hz to 1 MHz). In some instances, reactance at a lower frequency may be higher than resistance at a higher frequency, or may vary depending on the frequency.

E. Error Detection

An error detection algorithm can detect, for example, 14 different types of errors. Any number of errors or types of errors may be calculated by the detection algorithm. These errors are then classified and separated by a priority. For example, they may be classified on a scale from one to four, one is the highest priority and four is the lowest priority. Other scales and rankings can be used without departing from the scope of the invention. Thus, for example, Priority 1 can be associated with a problem with the connection/probe. Priority 2 can be associated with improper probe placement, Priority 3 can be associated with improper probe/tissue contact and finally Priority 4 can be associated with a movement artifact.

The error detection algorithm uses resistance and reactance values as detection criteria, from very well known types of errors identified during the human trials, through pattern recognition developed algorithms.

(a) After, for example, 10 spectra are obtained, the resistance and reactance average values are calculated for spectra without errors (e.g., using at least 5 spectra).

(b) For an average spectrum, a model algorithm can be applied to simplify the information obtained and to reduce noise.

### V. Model Calculation

The model calculation can be done in the following steps.
1. Spectrum segmentation
2. Semi circle curve fitting
3. Obtaining central resistances and reactances
4. Calculation of Tau
5. Obtaining central frequencies
6. Evaluating curve fitting error
   In some embodiments, a step (7) classification step may occur.

A further description of the model calculation steps are provided below.

1. The spectrum of resistance and reactance values may be segmented into 3 ranges:
Lower frequency range (9 first values)
Central frequency range (7 values)
Higher frequency range (9 last values)

In other embodiments, the resistance and reactance values may be segmented into any number of ranges. The ranges may be defined by the frequency values, or the number of samples collected at each frequency range. In some instances, only a low frequency range and high frequency range may be provided. In one example, a low frequency may be provided when the frequency is less than 10 kHz, and a high frequency may be provided when the frequency is greater than or equal to 10 kHz.

In the complex domain, the spectrum forms a shape of two semi circles, one at lower frequencies and another at higher frequencies. For both the lower and the higher frequency ranges, semi circles are fitted. The central frequency segment may contain values that are less useful for the semi circle curve fitting. The central frequency ranges may or may not have a fitted semi circle.

2. The lower and the higher frequency segments may each resemble a semi circle in the complex domain, made up by resistance/reactance points.

FIG. 7 is a graph which the central points contained in a Nyquist graph for each semi circle. The graph may show the reactance values as resistance varies. The original data may be provided and semi circles may be provided, fitted to the data. In some embodiments, two semi circles may be provided, one for high frequency and one for low frequency.

An initial semi circle is obtained using the minimum/maximum values of the resistance and reactance ranges. The resistance/reactance points may or may not be distributed over the semi circle very homogeneously, so for each point a weight is calculated. Points that are very close to each other get a lower weight, whereas points that don't have many other points nearby get a higher weight. This avoids obtaining a semi circle that only intersects a dense cluster of points but is not an accurate overall curve fit. The semi circle curve fitting algorithm iterates as follows.
Vary radius
Find best curve fit with current radius:
Vary center (x and y values)

This process is done continuously until acceptable thresholds are reached. The optimum is found by means of an error function. This function calculates the mean squared of the distance of the points to the fitted semi circle, taking the weights into account. If all the points are far away from the fitted semi circle, a large error value is obtained, whilst if they are very close, a small error value is obtained. Using this algorithm, the fitted semi circle will move towards the points. There are a number of conditions that must be met however. A number of key values that are calculated from the fitted semi circle are limited:
0.4<alfa<0.85
Rinf>0
Radius <50
Xc<1.1*Xmax

Alfa is a value linked with biological tissue, Rinf is the minimum point where the semi circle hits the Y axis (Reactance axis), Radius is the radius of the semi circle, Xmax is the maximum reactance value and Xc is the central value for the reactance (that should not exceed 110% of the maximum reactance value).

Instead of checking these conditions afterwards, they may be directly implemented in the algorithm to avoid obtaining unreasonable central values.

If the error increases instead of decreases, the search direction is inverted and the step size reduced, focusing in on the optimum values for the curve fit. When these step values become very small or the maximum number of calculation cycles is reached, the algorithm terminates, and a final curve fit error is obtained. In this final error the weights of the points are not considered.

The curve fitting algorithm described may be implemented in any curve fitting step of the method described herein. Alternatively, other curve fitting algorithms may be utilized. As previously described, any steps taken by a software or algorithm may be implemented by a tangible computer usable medium having a computer readable program code, logic, or instructions embedded therein, said computer readable program code, logic, or instructions adapted to be executed to implement the steps.

3. The central resistance is obtained as the centre point on the X axis of the semi circle. The central reactance is obtained as the centre point on the Y axis added by the radius of the semi circle. FIG. 7 shows that a central resistance can be provided for a high frequency (R_{H}) and a low frequency (R_{L}). Similarly, a central reactance can be provided for a high frequency (X_{H}) and a low frequency (X_{L}).

4. Tau (τ) may be calculated for each resistance and reactance value, and some key parameters directly derived of the fitted semi circle values. Tau may be a characteristic time constant. This value is directly linked to the central frequency. Tau may be calculated for each semi circle (i.e. Tau may be calculated for each frequency range). The calculation for Tau may be provided as previously discussed in equation (8).

5. The central frequency is directly calculated with the average of the Tau values: Fc=1/(2*PI*TAUavg). The central frequency may be calculated for each semi circle (i.e. central frequency may be calculated for each frequency range). In some instances a central frequency at low frequency and a central frequency at high frequency may be calculated.

If the reactance values are very small, and come very near to the X axis, the Tau cannot be calculated very well, and no central frequency can be obtained.

6. The final error of the semi circle curve fitting indicates if the curve fit was good, or was not. In the worst case, if the algorithm did not converge to a representative semi circle, resulting in a large error, the semi circle curve fit values are discarded.

So finally for each semi circle (corresponding with either the low or the high frequency range), the following central values are obtained:
Central resistance
Central reactance
Central frequency
   FIG. 7 shows the central points obtained in a Nyquist graph for each semi circle.

7. Classification

In some embodiments, the classification only uses the central reactance value at low frequency range.

The value is compared with the normal and abnormal levels.
Class 1: Xc(LF)<)(normal
Class 2: Xnormal <Xc(LF)<Xabnormal
Class 3: Xc(LF)>Xabnormal

(a) If an error spectrum occurs, it is stored in a variable to be displayed in the screen.

(b) If the average is done, information such as the time at which the average occurs, the number of spectra used in the average, the number of frequencies (e.g., 25), the errors for each spectrum, the classification, R_{L}, X_{L}, F_{L}, R_{H}, X_{H}, F_{H} values and the patient name are stored in a buffer. Finally, all these values are saved in a log file.

(c) Also, the reference and tissue signals, and the reference and tissue filtered signals are saved in a log file. In some embodiments, the data saved in the log file, or a subset thereof may form a physiological data profile. The log file may be stored in a memory within the system.

(d) Finally, a flag indicates that the screen will be updated with new values.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A system configured to determine characteristic parameter estimations of gastric impedance spectra comprising:
one or more monitoring device configured to measure at least one physiological parameter of a subject;
a memory including at least one physiological data profile with one or more electrical characteristic relating to a physical condition; and
a processor in communication with the monitoring device, wherein said processor:
receives the physiological parameter,
transforms the physiological parameter using a Cole model to one or more characteristic electrical value, and
compares said characteristic electrical value with the physiological data profile to determine whether the subject has the physical condition.

2. The system of claim 1 wherein the physiological parameter is an electrical impedance measurement.

3. The system of claim 1 wherein the characteristic electrical value includes at least one of: central resistance at low frequency, central resistance at high frequency, central reactance at low frequency, central reactance at high frequency, central frequency at low frequency, and central frequency at high frequency.

4. The system of claim 1 wherein the physical condition is at least one of the following: critically ill patients that have a healthy gastric mucosa, an ischemic or hypo-perfused mucosa, or an inflamed and damaged mucosa.

5. The system of claim 1 wherein the monitoring device measures a physiological parameter of a tissue of the subject, and the physical condition is healthy or hypoperfused tissue.

6. The system of claim 1 further comprising a display screen for displaying one or more result of said comparison.

7. The system of claim 1 further comprising a server configured to receive the physiological parameter from the monitoring device, and configured to communicate over a network with the processor.

8. A method for determining characteristic parameter estimation of gastric impedance spectra comprising:
receiving, at a processor, a first number of electrical measurements from a monitoring device configured to collect the electrical measurements from a tissue of a subject;
transforming, at the processor, the electrical measurements based on a model to a reduced number of characteristic parameters;
comparing the characteristic parameters with one or more profiles based on data representing gastric mucosa under different conditions.

9. The method of claim 8 wherein the model is a Cole model.

10. The method of claim 8 wherein the first number of electrical measurements exceeds 40.

11. The method of claim 8 wherein the reduced number of characteristic parameters is less than or equal to 6.

12. The method of claim 8 wherein the electrical measurements include gastric impedance measurements.

13. The method of claim 8 wherein the characteristic parameters include at least one of: central resistance at low frequency, central resistance at high frequency, central reactance at low frequency, central reactance at high frequency, central frequency at low frequency, and central frequency at high frequency.

14. The method of claim 8 wherein the different conditions may include at least one of: healthy gastric mucosa, gastric mucosa in patients undergoing cardiovascular surgery, and gastric mucosa in critically ill patients.

15. The method of claim 8 wherein collecting electrical measurements from the monitoring device includes the steps of:
starting electrical current tissue excitation at the tissue of the subject using one or more electrodes;
allowing a period of time to pass for a voltage signal to stabilize;
writing voltage data to a first in, first out (FIFO) memory buffer;
stopping electrical current tissue excitation;
stopping writing to the FIFO memory buffer; and
reading voltage data from the FIFO memory buffer.

16. The method of claim 15, wherein said collecting electrical measurement steps are repeated for multiple frequencies of tissue excitation.

17. The method of claim 16 wherein the voltage data is stored in a reference signal corresponding to a voltage drop across a reference resistor when an electrical current is excited, and a tissue signal corresponding to a differential voltage of a tissue that is measured at two central probe electrodes.

18. A tangible computer usable medium having a computer readable program code embedded therein, said computer readable program code adapted to be executed to implement a method for determining characteristic parameter estimation of gastric impedance spectra, the method comprising:
receiving a plurality of electrical measurements from a monitoring device configured to collect the electrical measurements from a tissue of a subject, wherein said electrical measurements include a tissue signal and a reference signal;
obtaining a phase value from for a tissue signal and reference signal pair;
obtaining an amplitude value for the tissue signal and reference signal pair;
calculating a resistance value and a reactance value from the phase value and the amplitude value.

19. The tangible computer readable medium of claim 18 wherein the method further comprises validating the electrical measurements by at least one of the following:
checking the tissue signal and the reference signal for value changes, or
checking the reference signal for a minimum amplitude.

20. The tangible computer readable medium of claim 18 wherein the method further comprising filtering the electrical measurements, thereby removing noise from the tissue signal and/or the reference signal.

21. The tangible computer readable medium of claim 18 wherein the phase value is calculated as a tissue phase minus a reference phase.

22. The tangible computer readable medium of claim 18 wherein the amplitude value is calculated as a tissue amplitude divided by a reference amplitude multiplied by a reference resistance.

23. The tangible computer readable medium of claim 18 wherein the resistance value is calculated by taking the real part of the polar coordinate value of the phase value and the amplitude value, and the reactance value is calculated by taking the imaginary part of the polar coordinate value of the phase value and the amplitude value.
